(19) United States
(12) Patent Application Publication
(10) Pub. No.: US 2022/0218456 A1
(43) Pub. Date: Jul. 14, 2022

(54) COMPOSITIONS, METHODS, AND KITS FOR SELECTION OF DONORS AND RECIPIENTS FOR IN VITRO FERTILIZATION

(71) Applicant: Vytelle, LLC, Hermiston, OR (US)

(72) Inventors: BRUNO VALENTE SANCHES, Southlake, TX (US); AMANDA FONSECA ZANGIROLAMO, Parana (BR); LUCIANO BONILLA, Hermiston, OR (US); MICHAEL D. BISHOP, Alexandria, SD (US)

(21) Appl. No.: 17/712,672

(22) Filed: Apr. 4, 2022

Related U.S. Application Data
(63) Continuation of application No. PCT/US20/54273, filed on Oct. 5, 2020.
(60) Provisional application No. 62/910,802, filed on Oct. 4, 2019.

(51) Int. Cl. A61D 19/04; A61D 7/00; C12Q 1/689

(52) U.S. Cl. CPC A61D 19/04; C12Q 2600/178; C12Q 1/689; A61D 7/00

(57) ABSTRACT

The present disclosure provides, inter alia, compositions, methods, and kits for improving the success rate of in vitro fertilization in small ruminants using microbiome profiles. According to some aspects, the present disclosure provides a method and kit for pairing female donor subjects with female recipient subjects, and for selecting female donor subjects. According to other aspects, the present disclosure provides a method of generating a microbiome database.

### CROSS REFERENCE TO RELATED APPLICATIONS

The present application is a continuation of PCT/US2020/054273, filed Oct. 5, 2020, which claims the benefit of U.S. Provisional Patent Application Ser. No. 62/910,802 filed on Oct. 4, 2019, which application is incorporated by reference herein in its entirety.

### BACKGROUND

In vitro fertilization (IVF) and embryo transfer techniques are commonly used in the production of small ruminant offspring, such as bovine calf production. IVF has some advantages over other production methods due to the frequency with which IVF aspirations can be performed and the ability to increase genetic diversity. For example, donor cow IVF aspirations can be performed every two weeks and semen from several different bulls can be used to fertilize harvested oocytes to produce a large number of offspring from a single cow. While embryo transfer techniques can produce about five or six embryos per collection every sixty days, IVF collections can produce about 20 oocytes per aspiration every two weeks, of which about 30% develop into viable embryos. In some circumstances, IVF can be used to produce over 50 calves from one cow in a single year. Furthermore, pregnant donor cows can still be used for oocyte collection until about day 100 to day 120 of pregnancy, making it possible to collect oocytes while still producing offspring from a high value cow.

There are, however, some disadvantages with using IVF techniques for small ruminant offspring production. The IVF procedure can be expensive and the follicle aspiration method used to collect oocytes is an invasive procedure that requires a skilled technician. Furthermore, even when performed properly, freshly transferred IVF embryos result in an average pregnancy rate of only about 50% for well managed recipient cows. Of the transfers that result in pregnancy, between about 6% to about 16% of pregnancies are lost. Thus, there is a need for compositions, methods and kits for improving the success rate of both initiation of pregnancy and maintenance of pregnancy when using IVF in order to maximize the efficiency of offspring production.

### SUMMARY OF THE INVENTION

According to some aspects, the present disclosure provides a method of pairing a female donor subject and a female recipient subject for in vitro fertilization comprising the steps of (1) obtaining microbial samples from each of the female donor subject and female recipient subject, wherein the microbial samples are obtained from one or more of the vaginal, uterine, and follicular fluid and/or surface; (2) identifying a microbiome profile for the vaginal, uterine, and follicular microbial samples from each of the female donor subject and female recipient subject; and (3) obtaining one or more oocytes or ovum from the female donor subject, fertilizing the one or more oocytes or ovum by in vitro fertilization, and implanting the fertilized embryo into the female recipient subject if the microbiome profile of the female donor subject and the microbiome profile of the female recipient subject indicate a high rate of initiation of pregnancy and high maintenance of pregnancy to term.

In some embodiments, the follicular microbiome profile of the female donor subject is indicative of quality and competence of an oocyte for in vitro production of embryos. In some embodiments, the vaginal and uterine microbiome profiles of the female recipient subject are indicative of high success of pregnancy after embryo transferred to the female recipient subject. In some embodiments, the microbiome profiles are generated by amplification and sequencing of ribosomal RNA. In some embodiments, the microbiome profiles comprise microbial taxonomy and relative microbial abundance. In some embodiments, the female donor subject and a female recipient subject are small ruminants. In some embodiments, the small ruminant is selected from the group consisting of cow, deer, sheep, goats, and buffalo. In some embodiments, the microbiome profile of the female donor subject and the microbiome profile of the female recipient subject indicate a rate of initiation of pregnancy of greater than 75% and maintenance of pregnancy of greater than 90%.

According to some aspects, the present disclosure provides a method of generating a microbiome database for pairing a female donor subject and a female recipient subject for in vitro fertilization comprising the steps of: (1) collecting microbial samples from a population of female donor subjects and a population of female recipient subjects, wherein the microbial samples are obtained from one or more of the vaginal, uterine, and follicular fluid and/or surface; (2) identifying a microbiome profile for one or more of the vaginal, uterine, and follicular microbial samples from each subject of the population of female donor subjects and the population of female recipient subjects; (3) obtaining one or more oocytes or ovum from one or more subjects in the population of female donor subjects, fertilizing the one or more oocytes or ovum by in vitro fertilization, and implanting the fertilized embryo into one or more subjects of the population of female recipient subjects; and (4) identifying an association of the microbiome profile, for one or more of the vaginal, uterine, and follicular microbial samples from one or more of the female donor subject and female recipient subject, with a high success rate of in vitro fertilization.

In some embodiments, the follicular microbiome profile of the female donor subject is indicative of quality and competence of an oocyte for in vitro production of embryos. In some embodiments, the vaginal and uterine microbiome profiles of the female recipient subject are indicative of high success of pregnancy after embryo transferred to the female recipient subject. In some embodiments, the microbiome profiles are generated by amplification and sequencing of ribosomal RNA. In some embodiments, the microbiome profiles comprise microbial taxonomy and relative microbial abundance. In some embodiments, a microbiome profile for one or more of the vaginal, uterine, and follicular microbial samples is identified for at least 300 female donor subjects and at least 300 female recipient subjects. In some embodiments, the microbiome profile for one or more of the vaginal, uterine, and follicular microbial samples is identified for female donor subjects and female recipient subjects stratified into one or more age ranges. In some embodiments, the population of female donor subjects and the population of female recipient subjects are small ruminants. In some embodiments, the small ruminant is selected from the group consisting of cow, deer, sheep, goats, and buffalo.

According to some aspects, the present disclosure provides a method of improving success rate of in vitro fertilization pregnancy comprising the steps of (1) obtaining microbial samples from one or more of a female donor subject and female recipient subject, wherein the microbial samples are obtained from one or more of the vaginal, uterine, and follicular fluid and/or surface; (2) identifying a microbiome profile for one or more of the vaginal, uterine, and follicular microbial samples from one or more of the female donor subject and female recipient subject; and (3) administering an intervention to one or more of the female donor subject and female recipient subject, wherein the intervention is effective to provide one or more female donor subject and female recipient subject with a microbiome profile associated with a high success rate of in vitro fertilization.

In some embodiments, the intervention is administered to the female donor subject and is effective to provide a microbiome profile of the follicular fluid and/or surface that is associated with high quality and competence of oocytes for in vitro production of embryos. In some embodiments, the intervention is administered to the female recipient subject and is effective to provide a microbiome profile of the vaginal and uterine fluid and/or surface that is associated with high success of pregnancy after embryo transfer. In some embodiments, the microbiome profiles are generated by amplification and sequencing of ribosomal RNA. In some embodiments, the microbiome profiles comprise microbial taxonomy and relative microbial abundance. In some embodiments, the female donor subjects and/or female recipient subjects are small ruminants. In some embodiments, the small ruminant is selected from the group consisting of cow, deer, sheep, goats, and buffalo. In some embodiments, the intervention is an antibiotic. In some embodiments, the intervention is one or more microbes (e.g., a probiotic). In some embodiments, the intervention is an undigestible nutritional composition that selectively stimulates the growth and activity of one or more host beneficial microbes (e.g., a prebiotic). In some embodiments, the intervention is administered to the vaginal or uterine cavity of the female donor subject or female recipient subject.

According to some aspects, the present disclosure provides a kit for pairing a female donor subject and a female recipient subject for in vitro fertilization comprising (1) one or more analytical tools for determining a microbiome profile of one or more of a follicular, vaginal, and uterine fluid and/or surface from the female donor subject and the female recipient subject; (2) a transmitter to communicate/connect a database of one or more microbiome profiles of one or more of a follicular, vaginal, and uterine fluid and/or surface; (3) a device for comparing the microbiome profile of one or more of a follicular, vaginal, and uterine fluid and/or surface from the female donor subject and the female recipient subject with the database of one or more microbiome profiles to identify which female donor subjects and the female recipient subjects have microbiome profiles indicative of a high success rate of in vitro fertilization; and (4) and instructions for use.

In some embodiments, the one or more analytical tools for determining a microbiome profile comprise reagents for amplification and sequencing of ribosomal RNA. In some embodiments, the microbiome profiles comprise microbial taxonomy and relative microbial abundance. In some embodiments, the female donor subjects and/or female recipient subjects are small ruminants. In some embodiments, the small ruminant is selected from the group consisting of cow, deer, sheep, goats, and buffalo. In some embodiments, the one or more analytical tools comprise a colorimetric assay. In some embodiments, the one or more analytical tools comprise a sequencing device.

In some embodiments, a method of selecting a female donor subject for in vitro fertilization comprising the steps of: (1) obtaining vaginal microbial samples from the female donor subject; (2) identifying a microbiome profile for the vaginal microbial samples from the female donor subject; and (3) obtaining one or more oocytes or ovum from the female donor subject for in vitro fertilization when the microbiome profile for the vaginal microbial samples indicative of the female donor subject will produce a high number of any selected from the group consisting of oocytes, ovum, and embryos.

A method of generating a microbiome database for selecting a female donor subject for in vitro fertilization comprising the steps of: (1) collecting vaginal microbial samples from a population of female donor subjects; (2) identifying a microbiome profile for the vaginal microbial samples from each subject of the population of female donor subjects; (3) obtaining oocytes or ovum from each subject in the population of female donor subjects and counting the number of any selected from the group consisting of oocytes, ovum and embryos produced; and (4) identifying an association of the microbiome profile for the vaginal microbial samples with production of a high number of any selected from the group consisting of oocytes, ovum and embryos.

A method of improving success rate of in vitro fertilization pregnancy comprising the steps of: (1) obtaining vaginal microbial samples from a female donor subject; (2) identifying a microbiome profile for the vaginal microbial samples; and (3) administering an intervention to the female donor subject, wherein the intervention is effective to provide the female donor subject with a microbiome profile associated with a high production of any selected from the group consisting of oocytes, ovum and embryos.

A kit for selecting a female donor subject for in vitro fertilization comprising: (1) one or more analytical tools for determining a microbiome profile of a vaginal microbial sample from the female donor subject; (2) a transmitter to communicate/connect a database of one or more microbiome profiles of vaginal microbial samples; (3) a device for comparing the microbiome profile of the vaginal microbial sample from the female donor subject and with the database of one or more microbiome profiles to identify which female donor subjects have microbiome profiles indicative of a high production of any selected from the group consisting of oocytes, ovum and embryos; and (4) and instructions for use.

### BRIEF DESCRIPTION OF THE DRAWINGS

The patent or application filed contains at least one drawing executed in color. Copies of this patent or patent application publication with color drawing(s) will be provided by the Office upon request and payment of the necessary fee.

FIG. 1 shows LEfSe at the species level. Linear discriminant analysis (LDA) combined with effect size measurements (LEfSe) revealed a list of bacteria that enable discrimination between the donors classified as Low or High in the vaginal swab samples. A p-value of <0.05 and a score ≥2.0 were considered significant in Kruskal-Wallis and pairwise Wilcoxon tests, respectively.

FIG. 2A-2D shows additional LEfSe at the species level. Histograms with the relative abundance of bacterial communities found in samples of vaginal swabs, present in higher quantity in the group of donors classified as Low in relation to High females. A p-value of <0.05 and a score ≥2.0 were considered significant in Kruskal-Wallis and pairwise Wilcoxon tests, respectively. The horizontal straight line in the panel indicates the group means, and the dotted line indicates the group medians. The representative bacterial species of the Low group, which showed a statistically significant higher amount than the females of High group, were Mogibacterium unclassified, Mobilibacterium unclassified, Planococcus unclassified, and Salinicoccus unclassified, present in histograms FIG. 2A-2D, respectively.

FIG. 3A-3D. shows additional LEfSe at the species level. Histograms with the relative abundance of bacterial communities found in samples of vaginal swabs, present in higher quantity in the group of donors classified as High in relation to Low females. A p-value of <0.05 and a score ≥2.0 were considered significant in Kruskal-Wallis and pairwise Wilcoxon tests, respectively. The horizontal straight line in the panel indicates the group means, and the dotted line indicates the group medians. The representative bacterial species of the High group, which showed a statistically significant higher amount than the females of Low group, were Anaerofustis unclassified, Bacteriodaceae unclassified, Abiotrophia unclassified, and Akkermansiaceae unclassified, present in histograms FIG. 3A-3D, respectively.

FIG. 4A-4D. shows additional LEfSe at the species level. Histograms with the relative abundance of bacterial communities found in samples of vaginal swabs, present in higher quantity in the group of donors classified as High in relation to Low females. A p-value of <0.05 and a score ≥2.0 were considered significant in Kruskal-Wallis and pairwise Wilcoxon tests, respectively. The horizontal straight line in the panel indicates the group means, and the dotted line indicates the group medians. The representative bacterial species of the High group, which showed a statistically significant higher amount than the females of Low group, were Liberibacter unclassified, Halomonas unclassified, Exiguobacterium unclassified, and Gemmatirosa unclassified, present in histograms FIG. 4A-4D, respectively.

FIG. 5A-5D shows additional LEfSe at the species level. Histograms with the relative abundance of bacterial communities found in samples of vaginal swabs, present in higher quantity in the group of donors classified as High in relation to Low females. A p-value of <0.05 and a score ≥2.0 were considered significant in Kruskal-Wallis and pairwise Wilcoxon tests, respectively. The horizontal straight line in the panel indicates the group means, and the dotted line indicates the group medians. The representative bacterial species of the High group, which showed a statistically significant higher amount than the females of Low group, were Pseudomonas litoralis, Prodoshia sp D12, Oscillatoriophycideae unclassified, and Negativibacillus massiliensis, present in histograms FIG. 5A-5D, respectively.

FIG. 6A-6C shows additional LEfSe at the species level. Histograms with the relative abundance of bacterial communities found in samples of vaginal swabs, present in higher quantity in the group of donors classified as High in relation to Low females. A p-value of <0.05 and a score ≥2.0 were considered significant in Kruskal-Wallis and pairwise Wilcoxon tests, respectively. The horizontal straight line in the panel indicates the group means, and the dotted line indicates the group medians. The representative bacterial species of the High group, which showed a statistically significant higher amount than the females of Low group, were Ruminiclostridium unclassified, Suicoccus unclassified, Pseudomonas saudimassiliensis, present in histograms FIG. 6A-6C, respectively.

FIG. 7 shows a series of steps for collection of follicular fluid samples according to some embodiments disclosed herein.

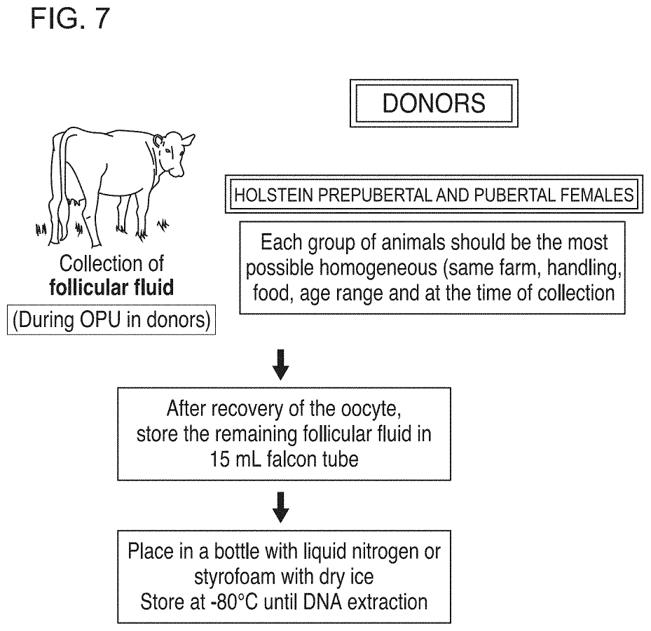

FIG. 8 shows a series of steps for collection of uterine/vaginal samples according to some embodiments disclosed herein.

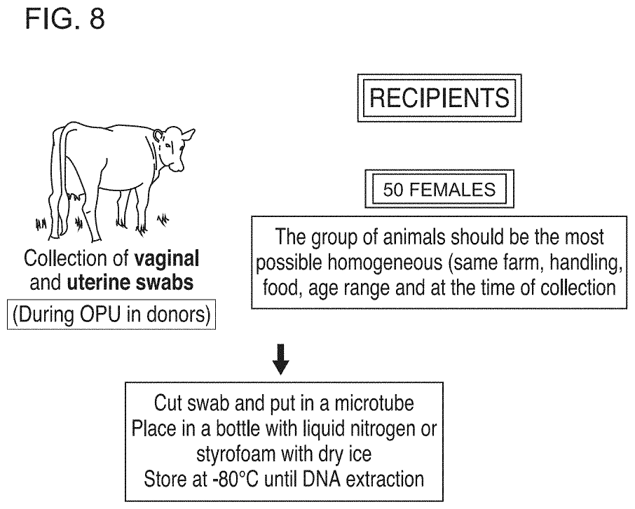

FIG. 9 shows a work flow for analyzing follicular, vaginal, and/or uterine samples.

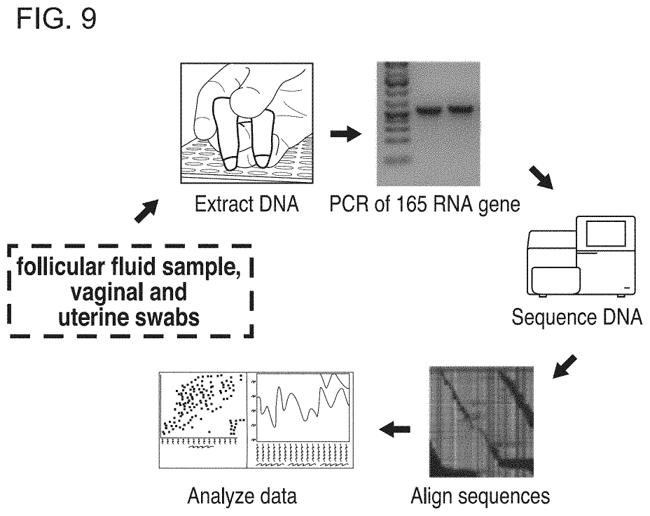

### DETAILED DESCRIPTION

The present disclosure provides compositions, methods, and kits for the selection of donor subjects and recipient subjects for in vitro fertilization. According to some aspects, the present disclosure provides a method utilizing a database of microbiome profiles from follicular fluid, vaginal tissue, and uterine tissue for complementary selection of female donors and female recipients to improve the efficiency of in vitro fertilization.

According to some aspects, the present disclosure provides a method of pairing a female donor subject and a female recipient subject for in vitro fertilization comprising the step of obtaining microbial samples from each of the female donor subject and female recipient subject. In some embodiments, the microbial samples are obtained from one or more of the vaginal, uterine, and follicular fluid and/or surface. In some embodiments, the method further comprises the step of identifying a microbiome profile for the vaginal, uterine, and follicular microbial samples from each of the female donor subject and female recipient subject. In some embodiments, the microbiome profiles are generated by sequencing of ribosomal RNA (rRNA) from the microbial samples. For example, the microbial profiles may be generated by sequencing the 16S ribosomal RNA gene. In some embodiments, the sequenced rRNA is used for taxonomic classification, such as phylum, order, class, family, genus, and species. In some embodiments, the sequenced rRNA is quantified to determine relative abundance of microbes present in the microbial samples. In some embodiments, the rRNA is PCR-amplified prior to sequencing.

According to some embodiments, the microbiome profiles obtained from the donor and/or recipient subject are compared to a database of microbiome profiles. In some embodiments, if the microbiome profile of the donor subject and recipient subject are associated with a high rate of initiation of pregnancy and high maintenance of pregnancy to term, then one or more oocytes or ovum are obtained from the donor subject, fertilized by in vitro fertilization, and implanted into the recipient subject.

In some embodiments, the follicular microbiome profile of the female donor subject is indicative of quality and competence of an oocyte for in vitro production of embryos. In some embodiments, the vaginal and uterine microbiome profiles of the female recipient subject are indicative of high success of pregnancy after embryo transferred to the female recipient subject.

In some embodiments, the female donor subject and a female recipient subject are small ruminants. As used herein, “ruminants” means any mammal that chews the cud regurgitated from its rumen, such as cow, deer, antelopes, sheep, goats, and buffalo and their relatives. In some embodiments, the small ruminant is selected from the group consisting of cow, deer, antelopes, sheep, goats, and buffalo. In some embodiments, the female donor subject and a female recipient subject are ungulate animals. As used herein, “ungulate animal” means any hoofed mammal. By way of non-limiting example, ungulate animals include odd-toed ungulates such as horses and rhinoceroses, and even-toed ungulates such as cattle, pigs, giraffes, camels, deer, and hippopotamuses, as well as sub-ungulates such as elephants. In some embodiments, the female donor subjects and female recipient subjects may be of any age.

In some embodiments, the microbiome profile of the female donor subject and the microbiome profile of the female recipient subject indicate a rate of initiation of pregnancy of greater than 50%. In some embodiments, the microbiome profile of the female donor subject and the microbiome profile of the female recipient subject indicate a rate of initiation of pregnancy of greater than 55%. In some embodiments, the microbiome profile of the female donor subject and the microbiome profile of the female recipient subject indicate a rate of initiation of pregnancy of greater than 60%. In some embodiments, the microbiome profile of the female donor subject and the microbiome profile of the female recipient subject indicate a rate of initiation of pregnancy of greater than 65%. In some embodiments, the microbiome profile of the female donor subject and the microbiome profile of the female recipient subject indicate a rate of initiation of pregnancy of greater than 70%. In some embodiments, the microbiome profile of the female donor subject and the microbiome profile of the female recipient subject indicate a rate of initiation of pregnancy of greater than 75%. In some embodiments, the microbiome profile of the female donor subject and the microbiome profile of the female recipient subject indicate a rate of initiation of pregnancy of greater than 80%. In some embodiments, the microbiome profile of the female donor subject and the microbiome profile of the female recipient subject indicate a rate of initiation of pregnancy of greater than 85%. In some embodiments, the microbiome profile of the female donor subject and the microbiome profile of the female recipient subject indicate a rate of initiation of pregnancy of greater than 90%.

In some embodiments, the microbiome profile of the female donor subject and the microbiome profile of the female recipient subject indicate a rate of maintenance of pregnancy of greater than 50%. In some embodiments, the microbiome profile of the female donor subject and the microbiome profile of the female recipient subject indicate a rate of maintenance of pregnancy of greater than 55%. In some embodiments, the microbiome profile of the female donor subject and the microbiome profile of the female recipient subject indicate a rate of maintenance of pregnancy of greater than 60%. In some embodiments, the microbiome profile of the female donor subject and the microbiome profile of the female recipient subject indicate a rate of maintenance of pregnancy of greater than 65%. In some embodiments, the microbiome profile of the female donor subject and the microbiome profile of the female recipient subject indicate a rate of maintenance of pregnancy of greater than 70%. In some embodiments, the microbiome profile of the female donor subject and the microbiome profile of the female recipient subject indicate a rate of maintenance of pregnancy of greater than 75%. In some embodiments, the microbiome profile of the female donor subject and the microbiome profile of the female recipient subject indicate a rate of maintenance of pregnancy of greater than 80%. In some embodiments, the microbiome profile of the female donor subject and the microbiome profile of the female recipient subject indicate a rate of maintenance of pregnancy of greater than 85%. In some embodiments, the microbiome profile of the female donor subject and the microbiome profile of the female recipient subject indicate a rate of maintenance of pregnancy of greater than 90%. In some embodiments, the microbiome profile of the female donor subject and the microbiome profile of the female recipient subject indicate a rate of maintenance of pregnancy of greater than 95%. In some embodiments, the microbiome profile of the female donor subject and the microbiome profile of the female recipient subject indicate a rate of maintenance of pregnancy of greater than 99%.

In some embodiments, the microbiome profile of the female donor subject indicates that the female donor subject will generate an increased number of embryos after oocyte fertilization. In some embodiments, the microbiome profile of the female donor subject indicates that the female donor subject will generate at least 1, at least 2, at least 3, at least 4, at least 5, at least 6, at least 7, at least 8, at least 9, at least 10, at least 15, at least 20, at least 25, at least 30, at least 40, at least 50, at least 60, at least 70, at least 80, at least 90, or at least 100 embryos on average per round of IVF.

According to some aspects, the present disclosure also provides a method of generating a microbiome database for pairing a female donor subject and a female recipient subject for in vitro fertilization. In some embodiments, the database is a collection of sequence information (for example, rRNA sequence information) that is organized so that is can be easily accessed, updated, and compared to other sequences, such as a computer database. In some embodiments, the sequence information in the database is obtained by collecting microbial samples from a population of female donor subjects and a population of female recipient subjects, wherein the microbial samples are obtained from one or more of the vaginal, uterine, and follicular fluid and/or surface. In some embodiments, the sequence information is associated with microbes from the microbial samples and is organized into a microbiome profile.

In some embodiments, the method of generating a microbiome database for pairing a female donor subject and a female recipient subject further comprises the step of identifying a microbiome profile for one or more of the vaginal, uterine, and follicular microbial samples from each subject of the population of female donor subjects and the population of female recipient subjects. In some embodiments, a microbiome profile for one or more of the vaginal, uterine, and follicular microbial samples is identified for at least 25 female donor subjects and at least 25 female recipient subjects. In some embodiments, a microbiome profile for one or more of the vaginal, uterine, and follicular microbial samples is identified for at least 50 female donor subjects and at least 50 female recipient subjects. In some embodiments, a microbiome profile for one or more of the vaginal, uterine, and follicular microbial samples is identified for at least 75 female donor subjects and at least 75 female recipient subjects. In some embodiments, a microbiome profile for one or more of the vaginal, uterine, and follicular microbial samples is identified for at least 100 female donor subjects and at least 100 female recipient subjects. In some embodiments, a microbiome profile for one or more of the vaginal, uterine, and follicular microbial samples is identified for at least 150 female donor subjects and at least 150 female recipient subjects. In some embodiments, a microbiome profile for one or more of the vaginal, uterine, and follicular microbial samples is identified for at least 200 female donor subjects and at least 200 female recipient subjects. In some embodiments, a microbiome profile for one or more of the vaginal, uterine, and follicular microbial samples is identified for at least 300 female donor subjects and at least 300 female recipient subjects.

In some embodiments, the method of generating a microbiome database for pairing a female donor subject and a female recipient subject further comprises the step of obtaining one or more oocytes or ovum from one or more subjects in the population of female donor subjects, fertilizing the one or more oocytes or ovum by in vitro fertilization, and implanting the fertilized embryo into one or more subjects of the population of female recipient subjects and then identifying an association of the microbiome profiles with a high success rate of in vitro fertilization.

In some embodiments, the follicular microbiome profile of the female donor subject is indicative of quality and competence of an oocyte for in vitro production of embryos. In some embodiments, the vaginal and uterine microbiome profiles of the female recipient subject are indicative of high success of pregnancy after embryo transferred to the female recipient subject. In some embodiments, the microbiome profile for one or more of the vaginal, uterine, and follicular microbial samples is identified for female donor subjects and female recipient subjects stratified into one or more age ranges.

According to some aspects, the present disclosure provides a method of improving success rate of in vitro fertilization pregnancy. In some embodiments, the method comprises that the steps of obtaining microbial samples from one or more of a female donor subject and female recipient subject, wherein the microbial samples are obtained from one or more of the vaginal, uterine, and follicular fluid and/or surface, identifying a microbiome profile for one or more of the vaginal, uterine, and follicular microbial samples from one or more of the female donor subject and female recipient subject, and administering an intervention to one or more of the female donor subject and female recipient subject, wherein the intervention is effective to provide one or more female donor subject and female recipient subject with a microbiome profile associated with a high success rate of in vitro fertilization.

In some embodiments, the success rate of in vitro fertilization pregnancy is measured by initiation of pregnancy. In some embodiments, the success rate of in vitro fertilization is an initiation of pregnancy greater than 50%. In some embodiments, the success rate of in vitro fertilization is an initiation of pregnancy greater than 55%. In some embodiments, the success rate of in vitro fertilization is an initiation of pregnancy greater than 60%. In some embodiments, the success rate of in vitro fertilization is an initiation of pregnancy greater than 65%. In some embodiments, the success rate of in vitro fertilization is an initiation of pregnancy greater than 70%. In some embodiments, the success rate of in vitro fertilization is an initiation of pregnancy greater than 75%. In some embodiments, the success rate of in vitro fertilization is an initiation of pregnancy greater than 80%. In some embodiments, the success rate of in vitro fertilization is an initiation of pregnancy greater than 85%. In some embodiments, the success rate of in vitro fertilization is an initiation of pregnancy greater than 90%.

In some embodiments, the success rate of in vitro fertilization is measured by maintaining pregnancy to term. In some embodiments, the success rate of in vitro fertilization is the maintenance of pregnancy at a rate greater than 50%. In some embodiments, the success rate of in vitro fertilization is the maintenance of pregnancy at a rate greater than 55%. In some embodiments, the success rate of in vitro fertilization is the maintenance of pregnancy at a rate greater than 60%. In some embodiments, the success rate of in vitro fertilization is the maintenance of pregnancy at a rate greater than 65%. In some embodiments, the success rate of in vitro fertilization is the maintenance of pregnancy at a rate greater than 70%. In some embodiments, the success rate of in vitro fertilization is the maintenance of pregnancy at a rate greater than 75%. In some embodiments, the success rate of in vitro fertilization is the maintenance of pregnancy at a rate greater than 80%. In some embodiments, the success rate of in vitro fertilization is the maintenance of pregnancy at a rate greater than 85%. In some embodiments, the success rate of in vitro fertilization is the maintenance of pregnancy at a rate greater than 90%. In some embodiments, the success rate of in vitro fertilization is the maintenance of pregnancy at a rate greater than 95%. In some embodiments, the success rate of in vitro fertilization is the maintenance of pregnancy at a rate greater than 99%.

In some embodiments, the intervention used to increase the success rate of in vitro fertilization is one or more of a probiotic, prebiotic, or antibiotic. In some embodiments, the intervention is administered directly to one or more of the follicle, vagina, and uterus of the donor and/or recipient subject. In some embodiments, the intervention is administered to the female donor subject and is effective to provide a microbiome profile of the follicular fluid and/or surface that is associated with high quality and competence of oocytes for in vitro production of embryos. In some embodiments, the intervention is administered to the female recipient subject and is effective to provide a microbiome profile of the vaginal and uterine fluid and/or surface that is associated with high success of pregnancy after embryo transfer.

According to some aspects, the present disclosure also provides a kit for pairing a female donor subject and a female recipient subject for in vitro fertilization. In some embodiments, the kit comprises one or more analytical tools for determining a microbiome profile of one or more of a follicular, vaginal, and uterine fluid and/or surface from the female donor subject and the female recipient subject. In some embodiments, the one or more analytical tools for determining a microbiome profile comprise an apparatus and/or reagents for amplification and/or sequencing of ribosomal RNA. In some embodiments, the one or more analytical tools comprise a portable real-time device for nucleotide sequencing and the reagents for use.

In some embodiments, the kit further comprises a transmitter to communicate/connect a database of one or more microbiome profiles to a device for comparing the microbiome profile of the female donor subject and the female recipient subject with the database of one or more microbiome profiles to identify which female donor subjects and the female recipient subjects have microbiome profiles indicative of a high success rate of in vitro fertilization.

In some embodiments, the kit comprises a database with a collection of sequence information (for example, rRNA sequence information) that is organized so that is can be easily accessed, updated, and compared to other sequences, such as a computer database. In some embodiments, the sequence information in the database is obtained by collecting microbial samples from a population of female donor subjects and a population of female recipient subjects, wherein the microbial samples are obtained from one or more of the vaginal, uterine, and follicular fluid and/or surface. In some embodiments, the sequence information is associated with microbes from the microbial samples and is organized into a microbiome profile. In some embodiments, the transmitter to communicate/connect with a database is wired or wireless. In some embodiments, the kit further comprises instructions for use.

According to some aspects, the present disclosure provides a method of selecting a female donor subject for in vitro fertilization comprising the steps of (1) obtaining vaginal microbial samples from the female donor subject; (2) identifying a microbiome profile for the vaginal microbial samples from the female donor subject; and (3) obtaining one or more oocytes or ovum from the female donor subject for in vitro fertilization when the microbiome profile for the vaginal microbial samples indicative of the female donor subject will produce a high number of any selected from the group consisting of oocytes, ovum and embryos.

In some embodiments, the vaginal microbiome profile of the female donor subject is associated with an average production of greater than 2, 3, 4, 5, 6, 7, 8, 9, or 10 of any selected from the group consisting of oocytes, ovum and embryos. In some embodiments, the microbiome profiles are generated by amplification and sequencing of ribosomal RNA. In some embodiments, the microbiome profiles comprise microbial taxonomy and relative microbial abundance. In some embodiments, the female donor subject is a small ruminant. In some embodiments, the small ruminant is selected from the group consisting of cow, deer, sheep, goats, and buffalo.

In some embodiments, the microbiome profile that is indicative of the female donor subject producing a high number of any selected from the group consisting of oocytes, ovum and embryos, comprises a lower relative abundance of one or more member of the genus of Mogibacterium, Mobilibacterium, Planococcus, and Salinicoccus; and/or a higher relative abundance of one or more of the genus of Anaerofustis, Bacteriodaceae, Abiotrophia, Akkermansiaceae, Liberibacter, Halomonas, Exiguobacterium, Gemmatirosa, Pseudomonas, Prodoshia, Oscillatoriophycideae, Negativibacillus, Ruminiclostridium, Suicoccus, and Pseudomonas. The relative abundances of microbes can be determined, according to certain embodiments, by linear discriminant analysis (LDA) combined with measurements of effective size (LEfSe), as described herein. For example, in one embodiment, the microbiome profile that is indicative of the female donor subject producing a high number of any selected from the group consisting of oocytes, ovum and embryos, is one that is absent one or more members of the genus of Mogibacterium, Mobilibacterium, Planococcus, and Salinicoccus; and/or that has present one or more of the genus of Anaerofustis, Bacteriodaceae, Abiotrophia, Akkermansiaceae, Liberibacter, Halomonas, Exiguobacterium, Gemmatirosa, Pseudomonas, Prodoshia, Oscillatoriophycideae, Negativibacillus, Ruminiclostridium, Suicoccus, and Pseudomonas.

In some embodiments, the microbiome profile that is indicative of the female donor subject producing a high number of any one selected from the group consisting of oocytes, ovum and embryos comprises a lower relative abundance of one or more of Mogibacterium, Mobilibacterium, Planococcus, and Salinicoccus unclassified; and/or a higher relative abundance of one or more of Anaerofustis, Bacteriodaceae, Abiotrophia, Akkermansiaceae, Liberibacter, Halomonas, Exiguobacterium, Gemmatirosa, Pseudomonas litoralis, Prodoshia sp D12, Oscillatoriophycideae, Negativibacillus massiliensis, Ruminiclostridium, Suicoccus, and Pseudomonas saudimassiliensis. For example, in some embodiments, the microbiome profile that is indicative of the female donor subject producing a high number of any one selected from the group consisting of oocytes, ovum and embryos is one that is absent one or more of Mogibacterium, Mobilibacterium, Planococcus, and Salinicoccus unclassified; and/or that has present one or more of Anaerofustis, Bacteriodaceae, Abiotrophia, Akkermansiaceae, Liberibacter, Halomonas, Exiguobacterium, Gemmatirosa, Pseudomonas litoralis, Prodoshia sp D12, Oscillatoriophycideae, Negativibacillus massiliensis, Ruminiclostridium, Suicoccus, and Pseudomonas saudimassiliensis.

According to some aspects, the present disclosure provides a method of generating a microbiome database for selecting a female donor subject for in vitro fertilization comprising the steps of (1) collecting vaginal microbial samples from a population of female donor subjects; (2) identifying a microbiome profile for the vaginal microbial samples from each subject of the population of female donor subjects; (3) obtaining oocytes or ovum from each subject in the population of female donor subjects and counting the number of any selected from the group consisting of oocytes, ovum and embryos produced; and (4) identifying an association of the microbiome profile for the vaginal microbial samples with production of a high number of any selected from the group consisting of oocytes, ovum and embryos. In some embodiments, the vaginal microbiome profile of the female donor subject is associated with an average production of greater than 2, 3, 4, 5, 6, 7, 8, 9, or 10 of any selected from the group consisting of oocytes, ovum and embryos. In some embodiments, the microbiome profiles are generated by amplification and sequencing of ribosomal RNA. In some embodiments, the microbiome profiles comprise microbial taxonomy and relative microbial abundance. In some embodiments, the female donor subject is a small ruminant. In some embodiments, the small ruminant is selected from the group consisting of cow, deer, sheep, goats, and buffalo. In some embodiments, the microbiome profile that is indicative of the female donor subject producing a high number of any selected from the group consisting of oocytes, ovum and embryos comprises a lower relative abundance of one or more member of the genus of Mogibacterium, Mobilibacterium, Planococcus, and Salinicoccus; and/or a higher relative abundance of one or more of the genus of Anaerofustis, Bacteriodaceae, Abiotrophia, Akkermansiaceae, Liberibacter, Halomonas, Exiguobacterium, Gemmatirosa, Pseudomonas, Prodoshia, Oscillatoriophycideae, Negativibacillus, Ruminiclostridium, Suicoccus, and Pseudomonas. In some embodiments, the microbiome profile that is indicative of the female donor subject producing a high number of any selected from the group consisting of oocytes, ovum and embryos comprises a lower relative abundance of one or more of Mogibacterium, Mobilibacterium, Planococcus, and Salinicoccus unclassified; and/or a higher relative abundance of one or more of Anaerofustis, Bacteriodaceae, Abiotrophia, Akkermansiaceae, Liberibacter, Halomonas, Exiguobacterium, Gemmatirosa, Pseudomonas litoralis Prodoshia sp D12, Oscillatoriophycideae, Negativibacillus massiliensis, Ruminiclostridium, Suicoccus, and Pseudomonas saudimassiliensis.

According to some aspects, the present disclosure provides a method of improving success rate of in vitro fertilization pregnancy comprising the steps of (1) obtaining vaginal microbial samples from a female donor subject; (2) identifying a microbiome profile for the vaginal microbial samples; and (3) administering an intervention to the female donor subject, wherein the intervention is effective to provide the female donor subject with a microbiome profile associated with a high production of any selected from the group consisting of oocytes, ovum and embryos.

In some embodiments, the vaginal microbiome profile of the female donor subject is associated with an average production of greater than 2, 3, 4, 5, 6, 7, 8, 9, or 10 of any selected from the group consisting of oocytes, ovum and embryos. In some embodiments, the microbiome profiles are generated by amplification and sequencing of ribosomal RNA. In some embodiments, the microbiome profiles comprise microbial taxonomy and relative microbial abundance. In some embodiments, the female donor subject is a small ruminant. In some embodiments, the small ruminant is selected from the group consisting of cow, deer, sheep, goats, and buffalo.

In some embodiments, the microbiome profile that is indicative of the female donor subject producing a high number of any selected from the group consisting of oocytes, ovum and embryos, comprises a lower relative abundance of one or more member of the genus of Mogibacterium, Mobilibacterium, Planococcus, and Salinicoccus; and/or a higher relative abundance of one or more of the genus of Anaerofustis, Bacteriodaceae, Abiotrophia, Akkermansiaceae, Liberibacter, Halomonas, Exiguobacterium, Gemmatirosa, Pseudomonas, Prodoshia, Oscillatoriophycideae, Negativibacillus, Ruminiclostridium, Suicoccus, and Pseudomonas. In some embodiments, the microbiome profile that is indicative of the female donor subject producing a high number of any selected from the group consisting of oocytes, ovum and embryos, comprises a lower relative abundance of one or more of Mogibacterium, Mobilibacterium, Planococcus, and Salinicoccus unclassified; and/or a higher relative abundance of one or more of Anaerofustis, Bacteriodaceae, Abiotrophia, Akkermansiaceae, Liberibacter, Halomonas, Exiguobacterium, Gemmatirosa, Pseudomonas litoralis, Prodoshia sp D12, Oscillatoriophycideae, Negativibacillus massiliensis, Ruminiclostridium, Suicoccus, and Pseudomonas saudimassiliensis.

In some embodiments, the intervention is an antibiotic. In some embodiments, the intervention is one or more microbes. In some embodiments, the intervention is a prebiotic. In some embodiments, the intervention is administered to the vaginal cavity of the female donor subject. In some embodiments, the intervention is one or more microbes comprising one or more of Anaerofustis, Bacteriodaceae, Abiotrophia, Akkermansiaceae, Liberibacter, Halomonas, Exiguobacterium, Gemmatirosa, Pseudomonas litoralis, Prodoshia sp D12, Oscillatoriophycideae, Negativibacillus massiliensis, Ruminiclostridium, Suicoccus, and Pseudomonas saudimassiliensis. In some embodiments, the intervention is effective to reduce the relative abundance of one or more of Mogibacterium, Mobilibacterium, Planococcus, and Salinicoccus unclassified and effective to increase relative abundance of one or more of Anaerofustis, Bacteriodaceae, Abiotrophia, Akkermansiaceae, Liberibacter, Halomonas, Exiguobacterium, Gemmatirosa, Pseudomonas litoralis, Prodoshia sp D12, Oscillatoriophycideae, Negativibacillus massiliensis, Ruminiclostridium, Suicoccus, and Pseudomonas saudimassiliensis.

According to some aspect, the present disclosure provides a kit for selecting a female donor subject for in vitro fertilization comprising: (1) one or more analytical tools for determining a microbiome profile of a vaginal microbial sample from the female donor subject; (2) a transmitter to communicate/connect a database of one or more microbiome profiles of vaginal microbial samples; (3) a device for comparing the microbiome profile of the vaginal microbial sample from the female donor subject and with the database of one or more microbiome profiles to identify which female donor subjects have microbiome profiles indicative of a high production of oocytes or ovum; and (4) and instructions for use. In some embodiments, the vaginal microbiome profile of the female donor subject is associated with an average production of greater than 2, 3, 4, 5, 6, 7, 8, 9, or 10 any selected from the group consisting of oocytes, ovum and embryos. In some embodiments, the microbiome profiles are generated by amplification and sequencing of ribosomal RNA. In some embodiments, the microbiome profiles comprise microbial taxonomy and relative microbial abundance. In some embodiments, the female donor subject is a small ruminant. In some embodiments, the small ruminant is selected from the group consisting of cow, deer, sheep, goats, and buffalo.

In some embodiments, the microbiome profile that is indicative of the female donor subject producing a high number of any selected from the group consisting of oocytes, ovum and embryos comprises a lower relative abundance of one or more member of the genus of Mogibacterium, Mobilibacterium, Planococcus, and Salinicoccus; and/or a higher relative abundance of one or more of the genus of Anaerofustis, Bacteriodaceae, Abiotrophia, Akkermansiaceae, Liberibacter, Halomonas, Exiguobacterium, Gemmatirosa, Pseudomonas, Prodoshia, Oscillatoriophycideae, Negativibacillus, Ruminiclostridium, Suicoccus, and Pseudomonas. In some embodiments, the microbiome profile that is indicative of the female donor subject producing a high number of any selected from the group consisting of oocytes, ovum and embryos, comprises a lower relative abundance of one or more of Mogibacterium, Mobilibacterium, Planococcus, and Salinicoccus unclassified; and/or a higher relative abundance of one or more of Anaerofustis, Bacteriodaceae, Abiotrophia, Akkermansiaceae, Liberibacter, Halomonas, Exiguobacterium, Gemmatirosa, Pseudomonas litoralis, Prodoshia sp D12, Oscillatoriophycideae, Negativibacillus massiliensis, Ruminiclostridium, Suicoccus, and Pseudomonas saudimassiliensis.

In some embodiments, the one or more analytical tools comprises a colorimetric assay. In some embodiments, the one or more analytical tools comprises a sequencing device.

The following examples are provided to further illustrate certain aspects of the present invention. These examples are illustrative only and are not intended to limit the scope of the invention in any way.

### EXAMPLES

### Example 1

### Materials and Methods

Animals and Classification of Donor Females

In the present study were used 47 Jersey (Bos taurus taurus) females aged 8 to 15 months, from properties located in Oregon—USA. The females were submitted to ovum pick-up—OPU, and the recovered oocytes destined for the in vitro embryo production (IVEP). At least 3 OPU procedures were performed per female. Then, from the number of embryos obtained during each OPU, the females were classified as Low, when the average embryo obtained during all procedures was between 0-1.9, or High-donors, when the average embryo obtained during all procedures was >4.0.

Collection of Vaginal Swabs

The swabs were removed from the packaging only at the time of collection. After proper containment of the animals, the external genitalia of the females were cleaned using tissue paper to remove all dirt and feces to prevent the contamination of the swab. The swabs were inserted 4 to 5 cm into the vagina, and full circles along the vaginal walls were performed for 20 seconds. Then, the swabs were cut, placed in cryovials correctly identified, and stored immediately in liquid nitrogen until the moment of DNA extraction.

DNA Extraction from Vaginal Swabs Samples

The genomic DNA from vaginal swabs were extracted using the PowerSoil DNA Isolation Kit (MO BIO Laboratory Inc., Carlsbad, Calif.) after disruption of the sample using a bead beater homogenizer (Mini-Beadbeater-8, Biospec Products), according to the manufacturer's protocol. The DNA concentration and purity of samples were measured using NanoDrop® ND-2000 (NanoDrop Technologies).

Long-Read DNA Sequencing and Bioinformatic Analysis

Targeted PCR amplification and high-throughput sequencing (amplicon sequencing) of 16S rRNA gene fragments is widely used to profile microbial communities. Current 16S profiling methods, however, do not provide sufficient taxonomic resolution and accuracy to perform species-level associative studies for specific conditions. This is due to the amplification and sequencing of only short regions of the 16S rRNA gene, usually providing only taxonomy at the family or genus level. However, longer sequences of the bacterial 16S rRNA gene could provide greater phylogenetic and taxonomic resolutions and advance knowledge of population dynamics within complex natural communities, moreover, provides species-level microbiome data. In this context, the sequencing method used was the Pacific Biosciences (PacBio) single molecule, real time (SMRT) sequencing based on DNA polymerization, a promising 3rd generation high-throughput technique.

Initially, from genomic DNA, duplicate and pooled V1-V9 amplicons were produced for the PacBio RS SMRT chip analysis. Samples were denatured (95° C., 5 min), followed by 28 cycles of denaturation (95° C., 45 s), annealing (55° C., 1 min), and extension (68° C., 2 min) with a final extension (68° C., 7 min). The PCR amplicons were visualized on agarose gel, purified using the QIAquick PCR purification kit (Qiagen, Valencia, Calif.). Post-amplification quality control was performed by on a Bioanalyzer (Agilent Technologies, Santa Clara, Calif., USA). Amplified DNA from the vaginal swab samples was then pooled in equimolar concentration.

SMRTbell libraries were prepared from the amplified DNA by blunt-ligation according to the manufacturer's instructions (Pacific Biosciences). Purified SMRTbell libraries from the pooled and barcoded vaginal samples were sequenced on a single PacBio Sequel cell. The samples were sequenced in the Sequel II system.

For the bioinformatics analysis, the Mothur software was used according to the method described by Kozich et al. (2013) [1] to align reads to the SILVA database. Then, the taxonomic classification was performed using the Ribosomal Database Project (RDP) 11.1.

Linear Discriminant Analysis (LDA) Coupled with Effect Size Measurements (LEfSe)

After the taxonomic classification, the data were analyzed in LEfSe, an algorithm for discovering and explaining high-dimension biomarkers that identify genomic characteristics (taxa, for example) that characterize the differences between two or more biological conditions (or classes) [2]. This approach can emphasize statistical significance, biological consistency, and the relevance of the effect, and identify differentially abundant characteristics consistent with biologically significant categories.

LEfSe first robustly identifies features that are statistically different between biological classes. It then performs additional tests to assess whether these differences are consistent with expected biological behavior. Initially, a Kruskal-Wallis (KW)-rank test [3] nonparametric factor sum is performed to detect characteristics with significant reference abundance concerning the class of interest; biological consistency is subsequently investigated using a set of peer tests between subclasses using the Wilcoxon rank-sum test (unpaired) [4,5]. Finally, LEfSe uses LDA [6] to estimate the effect size of each differentially abundant characteristic. A p-value of <0.05 and a score ≥2.0 were considered significant in Kruskal-Wallis and pairwise Wilcoxon tests, respectively.

### Results

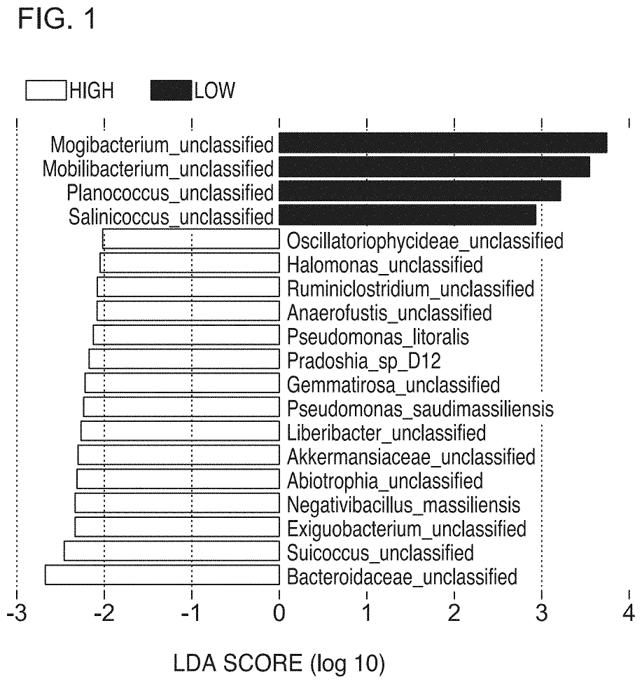

Through the linear discriminant analysis (LDA) combined with measurements of the effect size (LEfSe), it was possible to find a list containing 19 species of bacteria from the samples of vaginal swabs, which allow discrimination between female donors classified as Low and High embryo producers (FIG. 1).

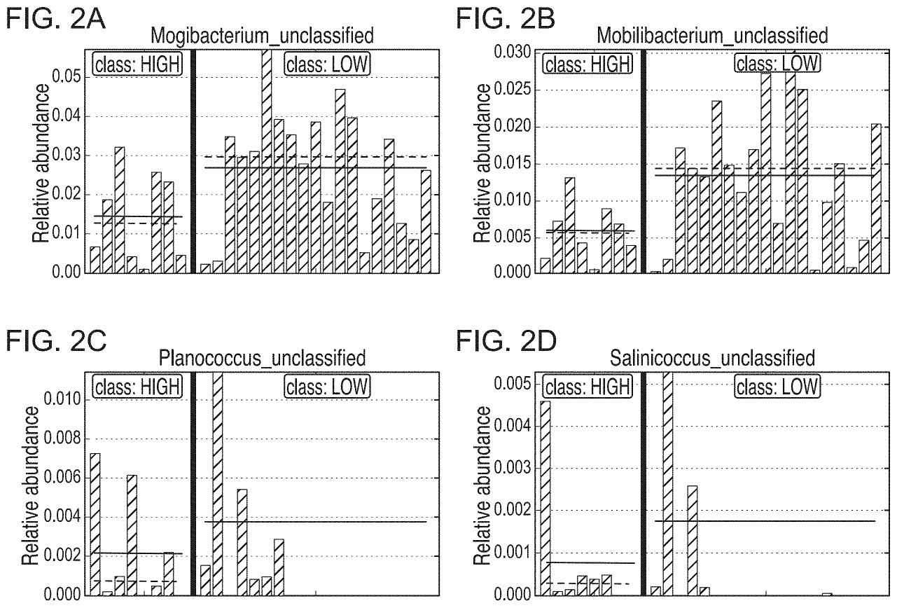

The species of bacteria representative of the group of females classified as Low (a total of 4 bacterial communities), and who had significantly greater relative abundance to the females classified as High-donors, are described in FIG. 2.

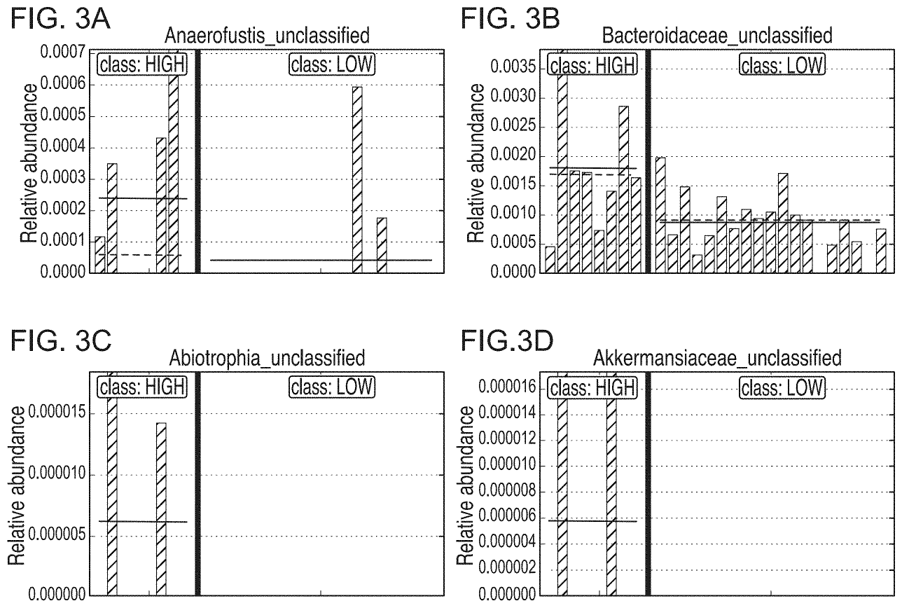

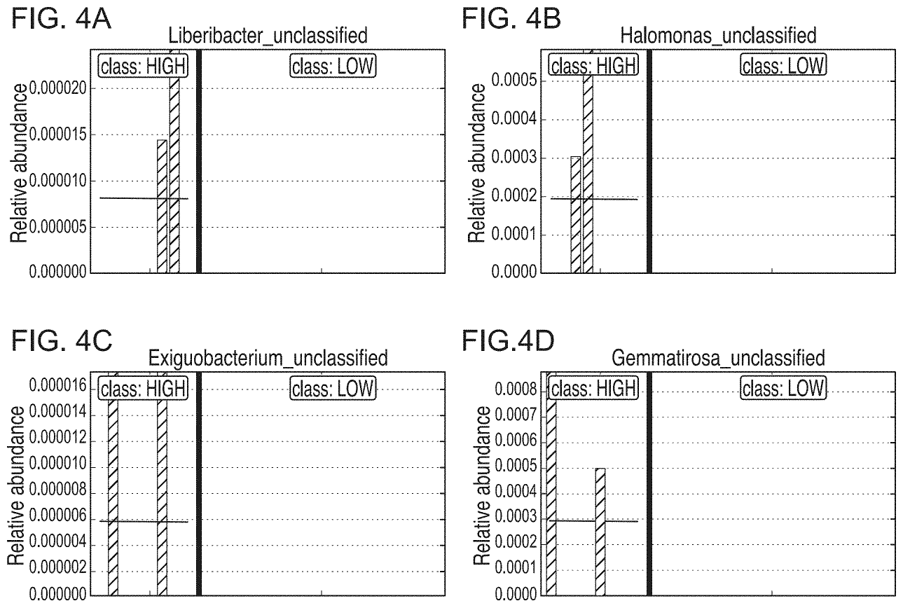

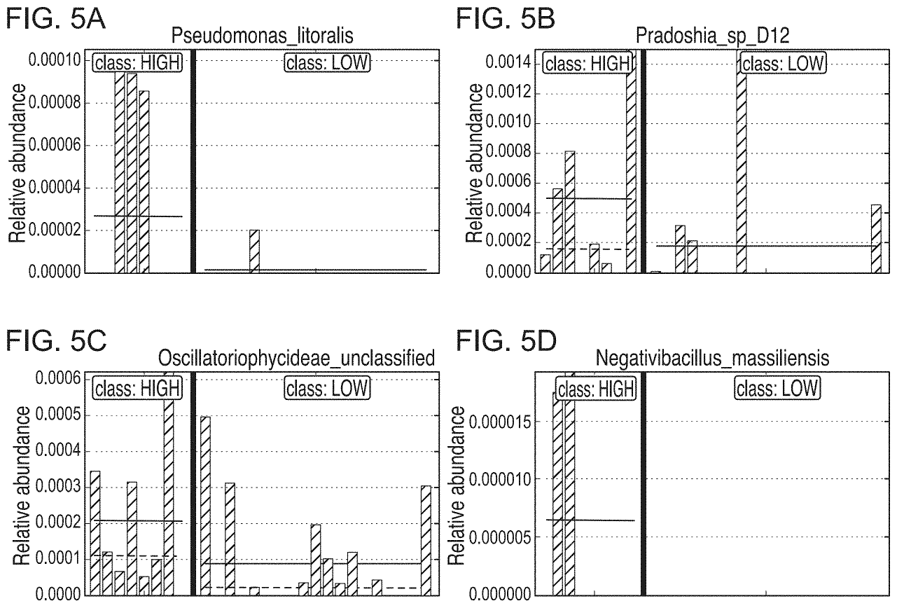

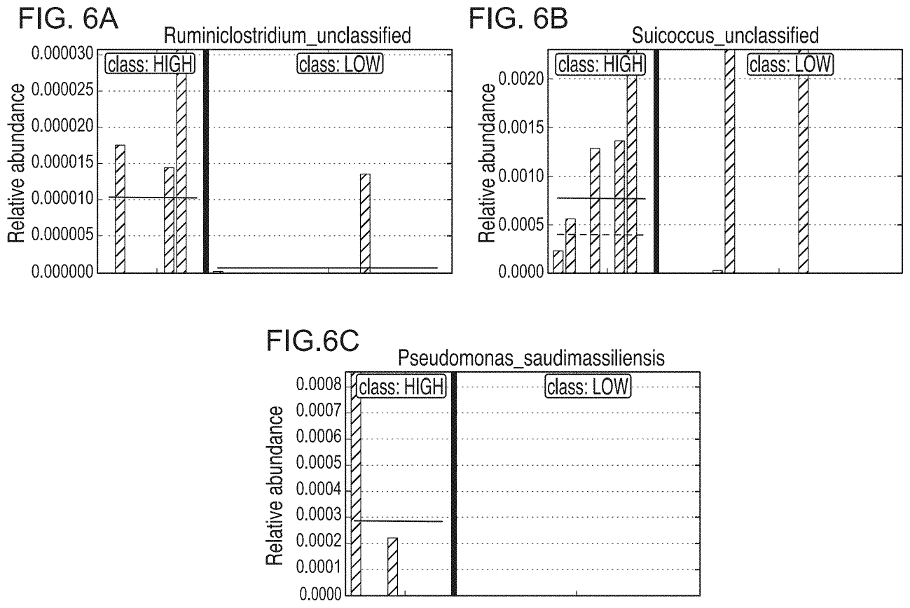

In contrast, the total of 15 bacterial communities, species of bacteria representative of the group of females classified as High, significantly greater relative abundance compared to females belonging to the Low group, are represented in FIGS. 3-6.

Based on the results obtained, it was possible to observe a distinct profile of bacterial species present in the vaginal microbiota of bovine females classified as Low or High donors of oocytes for the production of embryos in vitro. In this way, such bacterial communities can be better studied and analyzed later in a larger number of females, as possible targets for intervention or even for selecting oocyte donor females with better results in in vitro embryo production.

### Example 2

### Methods

Animals and Sampling

Collection of follicular fluid from donors: The follicular fluid will be collected from prepubertal and pubertal Holstein donors. After the OPU (as sterile as possible) the oocytes will be recovered, and the remaining follicular fluid will then be stored in sterile falcon tubes of 15 mL (in duplicates) correctly identified (follicular fluid—FL, animal code, age, and farm). The tubes will be stored immediately in liquid nitrogen and/or stored in a freezer −80 C until the time of DNA extraction.

Collection of vaginal and uterine swabs from recipients: During the embryo transfer procedure (Vytelle routine), vaginal and uterine swabs will be collected from 50 recipients. Swabs for this step may be the simplest as long as are sterile. The swabs will be removed from the packaging only at the time of collection. Firstly, the outside of the vulva will be cleaned with paper towels. The gloved veterinarian will open the lips and insert the swab into the vagina, taking care not to touch dirt or stool, promoting smear movements for approximately 20 seconds. Then the swab will be withdrawn, again taking care not to contaminate. The swab stem will be cut (with the aid of clean scissors) and placed in 1.5 mL (in duplicates) microtubes correctly identified (vaginal swab—VS, animal code, age, and farm). The microtubes will be stored immediately in liquid nitrogen and/or stored in a freezer −80 C until the moment of DNA extraction. Alternatively, the swab stem will be cut and placed into a solution for sample collection and nucleic acid stabilization for microbiome profile analysis, such as a system that is capable of lysing collected cells and stabilizing the sample for further analysis and evaluation. A non-limiting example of the type of collection system that might be suitable may be one similar to the commercially available Omnigene-Vaginal collection and stabilization kit (Catalog #: OMR-130), among other possible microbial collection and stabilization kits. In the same animals, uterine swabs will also be collected (for example Har-Vet™ 86 87 McCullough Double-Guarded Uterine Culture Swab, Spring Valley, Wis.) which was introduced to the cranial vagina. To avoid vaginal contamination of the swab, the plastic sheet covered pipette will be directed into the cervix. Inside the cervix, the plastic sheath (first layer of protection) will be ruptured, and the pipette will be manipulated through the cervix into the uterus. Once inside the uterus, the swab will then be advanced through the sealed plastic pipette (second layer of protection) exposing the sterile cotton swab to uterine secretion, promoting smear movements for approximately 20 seconds. The swab will be pulled inside the pipette and removed while the pipette will be maintained inside the uterus to avoid contamination by vaginal fluid. After removal of the uterine swab, the stem will be cut and placed into 1.5 mL microtubes (in duplicates) correctly identified (uterine swab—UTS, animal code, age, and farm). The microtubes will be stored immediately in liquid nitrogen and/or stored in a freezer −80 C until the moment of DNA extraction. Alternatively, the swab stem will be cut and placed into a commercially available solution for sample collection and nucleic acid stabilization, such as for example a system similar to the Omnigene-Vaginal collection and stabilization kit described above (Catalog #: OMR-130).

DNA Extraction

The genomic DNA from follicular fluid samples and uterine swabs will be extracted using the QIAamp DNA Mini kit (Qiagen). The genomic DNA from vaginal swabs will be extracted using the PowerSoil DNA Isolation Kit (MO BIO Laboratory Inc., Carlsbad, Calif.) after disruption of the sample using a bead beater homogenizer (Mini-Beadbeater-8, Biospec Products). The DNA concentrations of samples were measured using NanoDrop® ND-2000 (NanoDrop Technologies).

PCR Amplification of the V4 Hypervariable Region of Bacterial 16S rRNA Genes

To examine the bacterial community, the V4 region of the bacterial 16S ribosomal RNA (rRNA) gene will be PCR-amplified, and sequencing will be performed on the Illumina MiSeq platform. Samples that will be failed quality control will be excluded for taxonomic classification.

Data Analysis

Bacteria taxonomy (phylum, order, class, family, genus, and species) and relative abundance will be evaluated from sequences of data generated. Finally, all the results generated here will compose a database of microbiome profiles of donor and recipient females with different age ranges. Then, studies of the association between the bacterial profile and oocyte generation and, consequently, quality embryos (by evaluating rates obtained in the in vitro production of embryos), of the donors, or recipients that became pregnant after embryo transfer.

The embodiments described in this disclosure can be combined in various ways. Any aspect or feature that is described for one embodiment can be incorporated into any other embodiment mentioned in this disclosure. While various novel features of the inventive principles have been shown, described and pointed out as applied to particular embodiments thereof, it should be understood that various omissions and substitutions and changes may be made by those skilled in the art without departing from the spirit of this disclosure. Those skilled in the art will appreciate that the inventive principles can be practiced in other than the described embodiments, which are presented for purposes of illustration and not limitation. The disclosures of all patent and scientific literature cited herein are expressly incorporated by reference in their entirety.

### REFERENCES

1. Kozich J J, Westcott S L, Baxter N T, Highlander S K, Schloss P D. Development of a dual-index sequencing strategy and curation pipeline for analyzing amplicon sequence data on the MiSeq Illumina sequencing platform. Applied and Environmental Microbiology, 2013, 79(17):5112-20.
2. Segata N, Izard J, Walron L, Gevers D, Miropolsky L, Garrett W, Huttenhower C. Metagenomic Biomarker Discovery and Explanation. Genome Biology, 2011, 12(6):R60.
3. Kruskal W H, Wallis W A: Use of ranks in one-criterion variance analysis. J Am Stat Assoc, 1952, 47:583-621.
4. Wilcoxon F. Individual comparisons by ranking methods. Biometrics, 1945, 1:80-83.
5. Mann H B, Whitney D R. On a test of whether one of two random variables is stochastically larger than the other. Ann Math Stat, 1947, 18:50-60.
6. Fisher R A. The use of multiple measurements in taxonomic problems. Ann Eugenics, 1936, 7:179-188.

What is claimed is:

1. A method of pairing a female donor subject and a female recipient subject for in vitro fertilization comprising the steps of:
(1) obtaining microbial samples from each of the female donor subject and female recipient subject, wherein the microbial samples are obtained from one or more of the vaginal, uterine, and follicular fluid and/or surface;
(2) identifying a microbiome profile for the vaginal, uterine, and follicular microbial samples from each of the female donor subject and female recipient subject; and
(3) obtaining one or more oocytes or ovum from the female donor subject, fertilizing the one or more oocytes or ovum by in vitro fertilization, and implanting the fertilized embryo into the female recipient subject if the microbiome profile of the female donor subject and the microbiome profile of the female recipient subject indicate a high rate of initiation of pregnancy and high maintenance of pregnancy to term.

2. The method of embodiment 1, wherein the follicular microbiome profile of the female donor subject is indicative of quality and competence of an oocyte for in vitro production of embryos, or indicates an increased number of embryos produced by the female donor subject.

3. The method of embodiment 1, wherein the vaginal and uterine microbiome profiles of the female recipient subject are indicative of high success of pregnancy after embryo transferred to the female recipient subject.

4. The method of embodiment 1, wherein the microbiome profiles are generated by amplification and sequencing of ribosomal RNA.

5. The method of embodiment 1, wherein the microbiome profiles comprise microbial taxonomy and relative microbial abundance.

6. The method of embodiment 1, wherein the female donor subject and a female recipient subject are small ruminants.

7. The method of embodiment 6, wherein the small ruminant is selected from the group consisting of cow, deer, sheep, goats, and buffalo.

8. The method of embodiment 1, wherein the microbiome profile of the female donor subject and the microbiome profile of the female recipient subject indicate a rate of initiation of pregnancy of greater than 75% and maintenance of pregnancy of greater than 90%.

9. A method of generating a microbiome database for pairing a female donor subject and a female recipient subject for in vitro fertilization comprising the steps of:
(1) collecting microbial samples from a population of female donor subjects and a population of female recipient subjects, wherein the microbial samples are obtained from one or more of the vaginal, uterine, and follicular fluid and/or surface;
(2) identifying a microbiome profile for one or more of the vaginal, uterine, and follicular microbial samples from each subject of the population of female donor subjects and the population of female recipient subjects;
(3) obtaining one or more oocytes or ovum from one or more subjects in the population of female donor subjects, fertilizing the one or more oocytes or ovum by in vitro fertilization, and implanting the fertilized embryo into one or more subjects of the population of female recipient subjects; and
(4) identifying an association of the microbiome profile, for one or more of the vaginal, uterine, and follicular microbial samples from one or more of the female donor subject and female recipient subject, with a high success rate of in vitro fertilization.

10. The method of embodiment 9, wherein the follicular microbiome profile of the female donor subject is indicative of quality and competence of an oocyte for in vitro production of embryos, or indicates an increased number of embryos produced by the female donor subject.

11. The method of embodiment 9, wherein the vaginal and uterine microbiome profiles of the female recipient subject are indicative of high success of pregnancy after embryo transferred to the female recipient subject.

12. The method of embodiment 9, wherein the microbiome profiles are generated by amplification and sequencing of ribosomal RNA.

13. The method of embodiment 9, wherein the microbiome profiles comprise microbial taxonomy and relative microbial abundance.

14. The method of embodiment 9, wherein a microbiome profile for one or more of the vaginal, uterine, and follicular microbial samples is identified for at least 300 female donor subjects and at least 300 female recipient subjects.

15. The method of embodiment 9, wherein the microbiome profile for one or more of the vaginal, uterine, and follicular microbial samples is identified for female donor subjects and female recipient subjects stratified into one or more age ranges.

16. The method of embodiment 9, wherein the population of female donor subjects and the population of female recipient subjects are small ruminants.

17. The method of embodiment 16, wherein the small ruminant is selected from the group consisting of cow, deer, sheep, goats, and buffalo.

18. A method of improving success rate of in vitro fertilization pregnancy comprising the steps of:
(1) obtaining microbial samples from one or more of a female donor subject and female recipient subject, wherein the microbial samples are obtained from one or more of the vaginal, uterine, and follicular fluid and/or surface;
(2) identifying a microbiome profile for one or more of the vaginal, uterine, and follicular microbial samples from one or more of the female donor subject and female recipient subject; and
(3) administering an intervention to one or more of the female donor subject and female recipient subject, wherein the intervention is effective to provide one or more female donor subject and female recipient subject with a microbiome profile associated with a high success rate of in vitro fertilization.

19. The method of embodiment 18, wherein the intervention is administered to the female donor subject and is effective to provide a microbiome profile of the follicular fluid and/or surface that is associated with high quality and competence of oocytes for in vitro production of embryos, or an increased number of embryos produced by the female donor subject.

20. The method of embodiment 18, wherein the intervention is administered to the female recipient subject and is effective to provide a microbiome profile of the vaginal and uterine fluid and/or surface that is associated with high success of pregnancy after embryo transfer.

21. The method of embodiment 18, wherein the microbiome profiles are generated by amplification and sequencing of ribosomal RNA.

22. The method of embodiment 18, wherein the microbiome profiles comprise microbial taxonomy and relative microbial abundance.

23. The method of embodiment 18, wherein the female donor subjects and/or female recipient subjects are small ruminants.

24. The method of embodiment 23, wherein the small ruminant is selected from the group consisting of cow, deer, sheep, goats, and buffalo.

25. The method of embodiment 15, wherein the intervention is an antibiotic.

26. The method of embodiment 15, wherein the intervention is one or more microbes.

27. The method of embodiment 15, wherein the intervention is a prebiotic.

28. The method of embodiment 15, wherein the intervention is administered to the vaginal cavity of the female donor subject or female recipient subject.

29. A kit for pairing a female donor subject and a female recipient subject for in vitro fertilization comprising:
(1) one or more analytical tools for determining a microbiome profile of one or more of a follicular, vaginal, and uterine fluid and/or surface from the female donor subject and the female recipient subject;
(2) a transmitter to communicate/connect a database of one or more microbiome profiles of one or more of a follicular, vaginal, and uterine fluid and/or surface;
(3) a device for comparing the microbiome profile of one or more of a follicular, vaginal, and uterine fluid and/or surface from the female donor subject and the female recipient subject with the database of one or more microbiome profiles to identify which female donor subjects and the female recipient subjects have microbiome profiles indicative of a high success rate of in vitro fertilization; and
(4) and instructions for use.

30. The kit of embodiment 29, wherein the one or more analytical tools for determining a microbiome profile comprise reagents for amplification and sequencing of ribosomal RNA.

31. The kit of embodiment 29, wherein the microbiome profiles comprise microbial taxonomy and relative microbial abundance.

32. The kit of embodiment 29, wherein the female donor subjects and/or female recipient subjects are small ruminants.

33. The kit of embodiment 32, wherein the small ruminant is selected from the group consisting of cow, deer, sheep, goats, and buffalo.

34. The kit of embodiment 29, wherein the one or more analytical tools comprises a colorimetric assay.

35. The kit of embodiment 29, wherein the one or more analytical tools comprises a sequencing device.

36. A method of selecting a female donor subject for in vitro fertilization comprising the steps of:
(1) obtaining vaginal microbial samples from the female donor subject;
(2) identifying a microbiome profile for the vaginal microbial samples from the female donor subject; and
(3) obtaining one or more oocytes or ovum from the female donor subject for in vitro fertilization when the microbiome profile for the vaginal microbial samples indicative of the female donor subject will produce a high number of any selected from the group consisting of oocytes, ovum, and embryos.

37. The method of claim 36, wherein the vaginal microbiome profile of the female donor subject is associated with an average production of greater than 4 of any selected from the group consisting of oocytes, ovum, and embryos.

38. The method of claim 36, wherein the microbiome profiles are generated by amplification and sequencing of ribosomal RNA.

39. The method of claim 36, wherein the microbiome profiles comprise microbial taxonomy and relative microbial abundance.

40. The method of claim 36, wherein the female donor subject is a small ruminants.

41. The method of claim 40, wherein the small ruminant is selected from the group consisting of cow, deer, sheep, goats, and buffalo.

42. The method of claim 36, wherein the microbiome profile that is indicative of the female donor subject producing a high number of any selected from the group consisting of oocytes, ovum, and embryos comprises:
a lower relative abundance of one or more of Mogibacterium, Mobilibacterium, Planococcus, and Salinicoccus unclassified; and/or
a higher relative abundance of one or more of Anaerofustis, Bacteriodaceae, Abiotrophia, Akkermansiaceae, Liberibacter, Halomonas, Exiguobacterium, Gemmatirosa, Pseudomonas litoralis, Prodoshia sp D12, Oscillatoriophycideae, Negativibacillus massiliensis, Ruminiclostridium, Suicoccus, and Pseudomonas saudimassiliensis.

43. A method of generating a microbiome database for selecting a female donor subject for in vitro fertilization comprising the steps of:
(1) collecting vaginal microbial samples from a population of female donor subjects;
(2) identifying a microbiome profile for the vaginal microbial samples from each subject of the population of female donor subjects;
(3) obtaining oocytes or ovum from each subject in the population of female donor subjects and counting the number of any selected from the group consisting of oocytes, ovum, and embryos produced; and
(4) identifying an association of the microbiome profile for the vaginal microbial samples with production of a high number of any selected from the group consisting of oocytes, ovum, and embryos.

44. The method of claim 43, wherein the vaginal microbiome profile of the female donor subject is associated with an average production of greater than 4 of any selected from the group consisting of oocytes, ovum, and embryos.

45. The method of claim 43, wherein the microbiome profiles are generated by amplification and sequencing of ribosomal RNA.

46. The method of claim 43, wherein the microbiome profiles comprise microbial taxonomy and relative microbial abundance.

47. The method of claim 43, wherein the female donor subject is a small ruminants.

48. The method of claim 47, wherein the small ruminant is selected from the group consisting of cow, deer, sheep, goats, and buffalo.

49. The method of claim 43, wherein the microbiome profile that is indicative of the female donor subject producing a high number of any selected from the group consisting of oocytes, ovum, and embryos comprises:
a lower relative abundance of one or more of Mogibacterium, Mobilibacterium, Planococcus, and Salinicoccus unclassified; and/or
a higher relative abundance of one or more of Anaerofustis, Bacteriodaceae, Abiotrophia, Akkermansiaceae, Liberibacter, Halomonas, Exiguobacterium, Gemmatirosa, Pseudomonas litoralis, Prodoshia sp D12, Oscillatoriophycideae, Negativibacillus massiliensis, Ruminiclostridium, Suicoccus, and Pseudomonas saudimassiliensis.

50. A method of improving success rate of in vitro fertilization pregnancy comprising the steps of:
(1) obtaining vaginal microbial samples from a female donor subject;
(2) identifying a microbiome profile for the vaginal microbial samples; and
(3) administering an intervention to the female donor subject, wherein the intervention is effective to provide the female donor subject with a microbiome profile associated with a high production of any selected from the group consisting of oocytes, ovum, and embryos.

51. The method of claim 50, wherein the vaginal microbiome profile of the female donor subject is associated with an average production of greater than 4 of any selected from the group consisting of oocytes, ovum, and embryos.

52. The method of claim 50, wherein the microbiome profiles are generated by amplification and sequencing of ribosomal RNA.

53. The method of claim 50, wherein the microbiome profiles comprise microbial taxonomy and relative microbial abundance.

54. The method of claim 50, wherein the female donor subject is a small ruminant.

55. The method of claim 54, wherein the small ruminant is selected from the group consisting of cow, deer, sheep, goats, and buffalo.

56. The method of claim 50, wherein the microbiome profile that is indicative of the female donor subject producing a high number of any selected from the group consisting of oocytes, ovum, and embryos comprises:
a lower relative abundance of one or more of Mogibacterium, Mobilibacterium, Planococcus, and Salinicoccus unclassified; and/or
a higher relative abundance of one or more of Anaerofustis, Bacteriodaceae, Abiotrophia, Akkermansiaceae, Liberibacter, Halomonas, Exiguobacterium, Gemmatirosa, Pseudomonas litoralis, Prodoshia sp D12, Oscillatoriophycideae, Negativibacillus massiliensis, Ruminiclostridium, Suicoccus, and Pseudomonas saudimassiliensis.

57. The method of claim 50, wherein the intervention is an antibiotic.

58. The method of claim 50, wherein the intervention is one or more microbes.

59. The method of claim 50, wherein the intervention is a prebiotic.

60. The method of claim 50, wherein the intervention is administered to the vaginal cavity of the female donor subject.

61. The method of claim 50, wherein the intervention is one or more microbes comprising one or more of Anaerofustis, Bacteriodaceae, Abiotrophia, Akkermansiaceae, Liberibacter, Halomonas, Exiguobacterium, Gemmatirosa, Pseudomonas litoralis, Prodoshia sp D12, Oscillatoriophycideae, Negativibacillus massiliensis, Ruminiclostridium, Suicoccus, and Pseudomonas saudimassiliensis.

62. The method of claim 50, wherein the intervention is effective to reduce the relative abundance of one or more of Mogibacterium, Mobilibacterium, Planococcus, and Salinicoccus unclassified and effective to increase relative abundance of one or more of Anaerofustis, Bacteriodaceae, Abiotrophia, Akkermansiaceae, Liberibacter, Halomonas, Exiguobacterium, Gemmatirosa, Pseudomonas litoralis, Prodoshia sp D12, Oscillatoriophycideae, Negativibacillus massiliensis, Ruminiclostridium, Suicoccus, and Pseudomonas saudimassiliensis.

63. A kit for selecting a female donor subject for in vitro fertilization comprising:
(1) one or more analytical tools for determining a microbiome profile of a vaginal microbial sample from the female donor subject;
(2) a transmitter to communicate/connect a database of one or more microbiome profiles of vaginal microbial samples;
(3) a device for comparing the microbiome profile of the vaginal microbial sample from the female donor subject and with the database of one or more microbiome profiles to identify which female donor subjects have microbiome profiles indicative of a high production of any selected from the group consisting of oocytes, ovum, and embryos; and
(4) and instructions for use.

64. The kit of claim 63, wherein the vaginal microbiome profile of the female donor subject is associated with an average production of greater than 4 of any selected from the group consisting of oocytes, ovum, and embryos.

65. The kit of claim 63, wherein the microbiome profiles are generated by amplification and sequencing of ribosomal RNA.

66. The kit of claim 63, wherein the microbiome profiles comprise microbial taxonomy and relative microbial abundance.

67. The kit of claim 63, wherein the female donor subject is a small ruminant.

68. The kit of claim 67, wherein the small ruminant is selected from the group consisting of cow, deer, sheep, goats, and buffalo.

69. The method of claim 63, wherein the microbiome profile that is indicative of the female donor subject producing a high number of any selected from the group consisting of oocytes, ovum, and embryos comprises:
a lower relative abundance of one or more of Mogibacterium, Mobilibacterium, Planococcus, and Salinicoccus unclassified; and/or
a higher relative abundance of one or more of Anaerofustis, Bacteriodaceae, Abiotrophia, Akkermansiaceae, Liberibacter, Halomonas, Exiguobacterium, Gemmatirosa, Pseudomonas litoralis, Prodoshia sp D12, Oscillatoriophycideae, Negativibacillus massiliensis, Ruminiclostridium, Suicoccus, and Pseudomonas saudimassiliensis.

70. The kit of claim 63, wherein the one or more analytical tools comprises a colorimetric assay.

71. The kit of claim 73, wherein the one or more analytical tools comprises a sequencing device.

72. The method or kit of any of claims 36-71, wherein the higher or lower relative abundances are those determined by linear discriminant analysis (LDA) combined with measurements of effective size (LEfSe).
